(19) United States
(12) Patent Application Publication
(10) Pub. No.: US 2009/0186417 A1
(43) Pub. Date: Jul. 23, 2009

(54) Method for Determining the Oxygen Requirement of an Aqueous Solution for a Purification Process

(75) Inventors: Werner Arts, Berlin (DE); Berndt Martens, Bargteheide (DE)

Correspondence Address: CENTRAL COAST PATENT AGENCY, INC, 3 HANGAR WAY SUITE D, WATSONVILLE, CA, 95076, US

(73) Assignee: LAR PROCESS ANALYSERS AG, Berlin (DE)

(21) Appl. No.: 12/415,209

(22) Filed: Mar. 31, 2009

Related U.S. Application Data
(63) Continuation of application No. 10/526,259, filed on Mar. 1, 2005, now abandoned.

(30) Foreign Application Priority Data
Sep. 2, 2002 (DE) 10240140.0

(51) Int. Cl. G01N 33/18

(52) U.S. Cl. 436/62

(57) ABSTRACT

Method of determining the oxygen demand, TOD or COD, of an aqueous solution, in particular of waste water, for a clarification process, wherein a sample of the aqueous solution is decomposed by combustion, such that the combustion is carried out without the presence of a catalyst, at a temperature above 1150° C., in particular at 1200° C.

The invention relates to a method of determining the oxygen demand of an aqueous solution, in particular for a purification process.

A known method of determining the total content of organic carbon (TOC) in aqueous solutions, in particular waste water or also fresh water, is to subject the solution to high heat in a furnace and send the combustion gas to suitable detectors in order to establish the presence of compounds, the detection of which allows a conclusion to be drawn about the amount of organic carbon in the aqueous solution. Such combustion procedures are customarily carried out in the temperature range between about 600 and 850° C., maximally up to 950° C. The furnaces used for this purpose are operated, as a rule, with a mains voltage of 220 V and employ Ta wires as heating elements. Combustion occurs in the presence of a suitable catalyst and is therefore also called thermal-catalytic release.

The patent DE 44 12 778 C1 discloses a method of analysing a particle-containing aqueous sample, in particular for determining its organic carbon content, in which the sample is sprayed into a first combustion chamber which is then heated to about 1000° C. by an associated heating device, and the sample is thereby evaporated and combusted. After combustion is complete, the heating device in the first combustion chamber is turned off and the chamber is cooled down, and the combustion gases are passed through a catalyst in a second, horizontally oriented combustion chamber, where they are subjected to heat treatment in a range between 800° C. and 950° C. The combustion chamber in this arrangement is formed by an L-shaped quartz-glass tube. This secondary combustion chamber is filled with an oxidation catalyst, for example copper oxide.

Another method of determining TOC is known from EP 0 887 643 A1. In this method the sample is first raised from an initial temperature, below the simmering temperature of the water, to an evaporation temperature; then in a second step it is brought up to a considerably higher combustion temperature, preferably in the range between 800 and 1000° C. The use of a catalyst is not specified in this document; however, that one is needed is evident to the expert from the selected temperature range.

The document DE 199 23 139 A1 discloses a method and an apparatus for decomposing an aqueous solution in order to determine its carbon content in which the procedure includes a catalyst-free combustion at a temperature above 1000° C., in particular above 1200° C.

Another important quantity with respect to characterising the biochemical and/or chemical quality of water for processing purposes—in particular of sewage in preparation for clarification processes—is the amount of oxygen that will be required. This is in practice characterised by several quantities, of which the biochemical oxygen demand (BODₙ) has been used in practice for the longest time. Just like the so-called chemical oxygen demand (COD), which was not adopted for sewage analysis until considerably later, this parameter is determined by means of a complicated, several-stage biochemical or chemical decomposition procedure. A short-term determination is decidedly difficult, the measurement results are poorly reproducible and the microorganisms are vulnerable to toxic matrix components, pH shifts and the accumulation of inhibitory metabolic products. In contrast, the standardised methods of COD determination provide excellently reproducible results, but cannot be well automated and make severe demands regarding protection of the workers.

A parameter for quantifying the organic load of waste water that is advantageous from this point of view is the total oxygen demand (TOD), which is measured by a procedure including thermal oxidation by combustion of the sample in a high-temperature reactor. This involves detection of not only the organic substances contained in the sample but also, in part, other organic compounds. In many investigations a good correlation has been found between COD and TOD, so that recently more consideration has been given to replacing the quantity COD by the quantity TOD as a quality parameter for water/sewage.

In the paper by W.-J. Becker “Zur Bestimmung des totalen Sauerstoff-Bedarfs (TOD)”, X. f. Wasser- und Abwasser-Forschung, 12, 5/1979, 196 the procedure for TOD measurement is described in detail, and a survey of the main parameters of the most important commercially available COD and TOD waste-water analysis devices is given. From this survey it can be seen that the oxidation temperatures are mostly 900° C., in some cases also 850° C. The author of the publications made measurements at 1100° C. in a horizontal tube furnace.

The document JP-B-977-26111 describes a combined TOC and TOD measurement in which the sample is treated in a combustion chamber at 500° C.

During practical employment of the known catalytic thermal decomposition procedures, i.e. in the routine operation of water-treatment plants, sewage works and the like, organizational problems have arisen regarding the manipulation of the catalysts employed, which can be damaged by certain sample components, in particular metal ions or complexes, and hence must be replaced periodically. Failure to carry out such replacements can, in some circumstances, cause erroneous measurements and hence mismanagement of the processes controlled by the results of these measurements.

It is the objective of the invention to disclose a method of this generic kind that is simpler to put into operation.

This objective is achieved by a method with the characteristics given in Claim 1.

The invention, accordingly, includes the essential idea of departing from the thermal-catalytic manner of decomposition and performing this operation entirely thermally, without using a catalyst. It further includes the idea that for this purpose the combustion temperature is increased, to values above 1150° C. and specifically to a value of approximately 1200° C.

Elimination of the catalyst required for the known methods is advantageous not only with regard to costs—which vary depending on the nature of the catalyst—but primarily in that it produces the desired facilitation of the procedural organization, because there is no longer any need to keep a store of appropriate catalyst material available or to schedule and execute a periodic renewal of such material, and hence a more reliabile operation of the plant once it is no longer possible to mismanage such material. These advantages far outweigh the disadvantage of a slightly higher energy consumption during operation of the plant, owing to the higher combustion temperature.

It is particularly advantageous to carry out the method in an elongated, substantially vertically oriented reaction chamber. This offers the especially advantageous opportunity to remove salts contained in the sample at the lower end of the reaction chamber and thus prevent “salinification” of the reaction chamber. This saves a considerable amount of maintenance work and, of course, the associated costs.

1. Method of determining the oxygen demand, TOD or COD, of an aqueous solution, in particular of waste water, in particular for a purification process, wherein a sample of the aqueous solution is decomposed by combustion,
characterized in that
the combustion is carried out without the presence of a catalyst in an elongated and substantially vertically oriented reaction chamber, to the upper region of which the aqueous solution is conducted, at a temperature above 1150° C., in particular at 1200° C.

2. Method according to claim 1,
characterized in that salts contained in the sample are removed at the lower end of the reaction chamber.
